(19) United States
(12) Patent Application Publication
(10) Pub. No.: US 2020/0400915 A1
(43) Pub. Date: Dec. 24, 2020

(54) DRIVE UNIT, IMAGE PICKUP APPARATUS, AND ENDOSCOPE

(71) Applicant: OLYMPUS CORPORATION, Tokyo (JP)

(72) Inventor: Tadashi ITO, Tokyo (JP)

(73) Assignee: OLYMPUS CORPORATION, Tokyo (JP)

(21) Appl. No.: 17/011,161

(22) Filed: Sep. 3, 2020

Related U.S. Application Data
(63) Continuation of application No. PCT/JP2018/008510, filed on Mar. 6, 2018.

(51) Int. Cl. G02B 7/04; H02K 11/215; H02K 41/035; A61B 1/00; A61B 1/05

(52) U.S. Cl. CPC G02B 7/04; H02K 11/215; G02B 23/2484; A61B 1/00188; A61B 1/05; H02K 41/0356

(57) ABSTRACT

A drive unit includes: a fixed barrel; a movable barrel; a magnet unit including a first magnet, a second magnet, and a third magnet being polarized in an optical axis direction L such that magnetic poles of the third magnet have the same polarities as a magnetic pole of the first magnet at an outer side in the radial direction and a magnetic pole of the second magnet at an outer side in the radial direction; a coil unit including a first coil and a second coil; and a movable range restricting member.

### CROSS REFERENCE TO RELATED APPLICATION

This application is a continuation application of PCT/JP2018/008510 filed on Mar. 6, 2018, the entire contents of which are incorporated herein by this reference.

### BACKGROUND OF THE INVENTION

### 1. Field of the Invention

The present invention relates to a drive unit capable of moving a movable barrel in an optical axis direction by applying a current which flows through a coil unit to a magnetic field generated by a magnet unit disposed on the movable barrel, an image pickup apparatus, and an endoscope.

### 2. Description of the Related Art

There has been well known an image pickup apparatus which includes a movable barrel which holds an optical system for forming an optical image of an object in the movable barrel, and is movable in an advancing and retracting manner in an optical axis direction of the optical system in a fixed barrel to switch a focal point of a photographic object. The image pickup apparatus is disposed in an insertion section of an endoscope, for example.

Also well-known is a configuration where a drive unit, for example, a voice coil motor which an image pickup apparatus includes is used for moving a movable barrel in a fixed barrel. For example, Japanese Patent Application Laid-Open Publication No. 2015-114651 discloses such a configuration.

A voice coil motor disclosed in Japanese Patent Application Laid-Open Publication No. 2015-114651 has four magnet units on an outer peripheral surface of a movable barrel at an equal interval in a circumferential direction of the outer peripheral surface, wherein each magnet unit is configured by two magnets which are polarized such that magnetic poles of two magnets are arranged differently from each other in a radial direction of the movable barrel.

A coil unit configured by two electromagnetic coils are wound around an outer peripheral surface of a fixed barrel at positions which face two magnets in a state where two electromagnetic coils are disposed adjacently to each other in an optical axis direction, and two electromagnetic coils are configured such that current directions of two electromagnetic coils are inverted from each other in an energized state.

With such a configuration, when a current is supplied to the coil unit, the movable barrel is moved in an optical axis direction by a magnetic field generated by the magnetic unit with the supply of the current due to Fleming's left-hand rule.

Further, to allow the movement of the movable barrel in the fixed barrel, a gap is formed between an outer peripheral surface of the movable barrel and an inner peripheral surface of the fixed barrel. The plurality of magnet units disposed on the movable barrel generate magnetic fields in a plurality of directions in a radial direction.

Also well-known is a configuration of a voice coil motor where a plurality of magnet units are disposed on an outer peripheral surface of a movable barrel, and an energizing plate which faces any one of the plurality of magnet units in a radial direction and generates an attracting force with respect to the facing magnet unit is disposed outside a coil unit in the radial direction.

With such a configuration, due to an attracting force of the energizing plate, an outer peripheral surface of the movable barrel butts against an inner peripheral surface of a fixed barrel on an energizing plate side and hence, backlash can be prevented.

Also well-known is a configuration of a voice coil motor where a Hall device which is a position detection member is disposed in a fixed barrel such that the Hall device faces any one of a plurality of magnet units disposed on an outer periphery of a movable barrel in a radial direction, and a magnetic field of the magnet unit which the Hall device faces is detected by the Hall device, thereby detecting a position of the movable barrel in the fixed barrel.

When an image pickup apparatus which includes a voice coil motor is used in an endoscope, to decrease a diameter of an insertion section of the endoscope, it is inevitably necessary to miniaturize the image pickup apparatus, that is, to decrease a diameter of the image pickup apparatus. As a result, it is also inevitably necessary to miniaturize the voice coil motor, that is, to decrease a diameter of the voice coil motor.

It has been known that assemblability of a voice coil motor is poor when mounting magnet units on an outer peripheral surface of a movable barrel because of a phenomenon that magnets jump out due to the repulsive force generated between magnets in the magnet unit. To decrease a diameter of the voice coil motor, it is desirable that the voice coil motor have the configuration where the movable barrel is movable with a small drive loss by improving assemblability of the magnet unit.

More specifically, it is desirable for a voice coil motor to have a configuration where, regardless of a position to which a movable barrel is moved, one of magnets which form a magnet unit constantly faces one of electromagnetic coils which constitute a coil unit in a radial direction, and the other magnet constantly faces the other electromagnetic coil in the radial direction.

### SUMMARY OF THE INVENTION

A drive unit according to an aspect of the present invention includes: a fixed barrel having a barrel shape; a movable barrel configured to hold an optical system which forms an optical image of an object, the movable barrel being movable in the fixed barrel in an optical axis direction of the optical system; a plurality of magnet units disposed on an outer periphery of the movable barrel at an equal interval in a circumferential direction, each of the plurality of magnet units being configured by three or more magnets disposed adjacently to each other in the optical axis direction; a first magnet and a second magnet in each of the magnet units, the first magnet and the second magnet being polarized such that magnetic poles of the first magnet and magnetic poles of the second magnet are arranged differently from each other in a radial direction of the movable barrel orthogonal to the optical axis direction, and being disposed so as not to be adjacent to each other in the optical axis direction; a third magnet disposed adjacently to at least one of the first magnet and the second magnet in the optical axis direction and being polarized in the optical axis direction such that magnetic poles of the third magnet have same polarities as a magnetic pole of the first magnet at an outer side in the radial direction and a magnetic pole of the second magnet at the outer side in the radial direction; a coil unit wound around an outer periphery of the fixed barrel, the coil unit being configured to move the movable barrel in the optical axis direction by generating a magnetic field applied to the magnet units; a first coil and a second coil in the coil unit, a current direction of the first coil and a current direction of the second coil being inversed from each other in an energized state, the first coil facing the first magnet in the radial direction, and the second coil facing the second magnet in the radial direction; and a movable range restricting member configured to allow both end portions of the movable barrel to come into contact with the movable range restricting member in the optical axis direction, the movable range restricting member being configured to define a state where the first magnet faces the first coil and a state where the second magnet faces the second coil regardless of movement of the movable barrel in the optical axis direction.

An image pickup apparatus according to another aspect of the present invention includes the drive unit.

An endoscope according to still another aspect of the present invention includes the image pickup apparatus.

### BRIEF DESCRIPTION OF THE DRAWINGS

FIG. 1 is a view showing an external appearance of an endoscope which includes an image pickup apparatus having a drive unit according to a first embodiment;

FIG. 2 is a front view of the image pickup apparatus which is disposed in a distal end portion of an insertion section of the endoscope shown in FIG. 1;

FIG. 3 is a cross-sectional view of the image pickup apparatus taken along a line III-III in FIG. 2;

FIG. 4 is a cross-sectional view of a drive unit taken along a line IV-IV in FIG. 3;

FIG. 5 is a perspective view schematically showing a movable barrel on which magnet units are disposed in the image pickup apparatus shown in FIG. 3;

FIG. 6 is a perspective view of the movable barrel shown in FIG. 5 as viewed from a direction VI in FIG. 5;

FIG. 7 is a side view showing a configuration where a rotation stopper is disposed on an outer peripheral surface of the movable barrel shown in FIG. 5;

FIG. 8 is a partial cross-sectional view showing one example of a magnet unit disposed on an outer peripheral surface of a movable barrel in a drive unit according to a second embodiment;

FIG. 9 is a partial cross-sectional view showing a modification where a position defining surface shown in FIG. 8 is formed on a protruding portion formed on the outer peripheral surface of the movable barrel;

FIG. 10 is a partial cross-sectional view showing a modification where the position defining surface shown in FIG. 8 is formed on a stepped portion against which a third magnet butts;

FIG. 11 is a partial cross-sectional view showing one example of a magnet unit disposed on an outer peripheral surface of a movable barrel in a drive unit according to a third embodiment together with a magnetic member and a Hall device; and

FIG. 12 is a partial cross-sectional view showing a modification where a coil unit is configured by five magnets.

### DETAILED DESCRIPTION OF THE PREFERRED EMBODIMENTS

Hereinafter, embodiments of the present invention are described with reference to drawings. The drawings are schematic views. Note that, of course, a relationship between a thickness and a width of each member, a ratio between thicknesses of respective members and the like differ from the corresponding relationships of actual members. It goes without saying that portions of the drive unit, the image pickup apparatus, and the endoscope may be described with different size relationships or different ratios between the drawings.

### First Embodiment

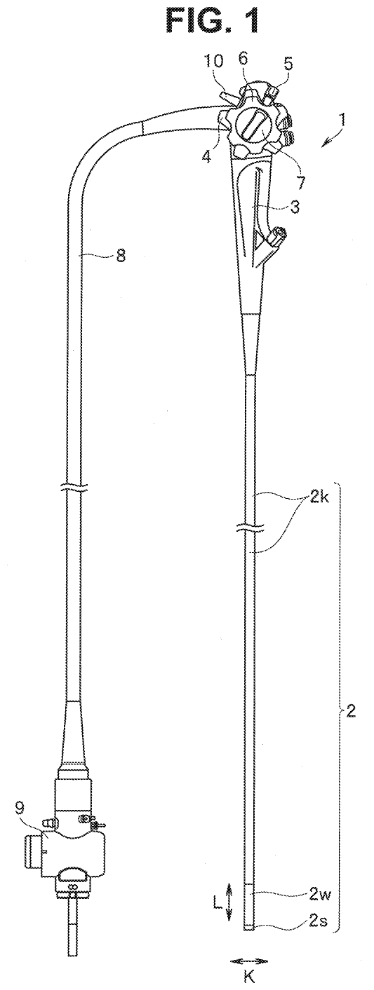

FIG. 1 is a view showing an external appearance of an endoscope which includes an image pickup apparatus having a drive unit according to a first embodiment.

As shown in FIG. 1, the endoscope 1 mainly includes: an insertion section 2 which is inserted into a subject; an operation section 3 which is connected to a proximal end side of the insertion section 2; a universal cord 8 which extends from the operation section 3; and a connector 9 which is disposed at an extending end of the universal cord 8. The endoscope 1 is electrically connected to external apparatuses such as a controller and an illumination apparatus via the connector 9.

The operation section 3 includes: a vertical bending operation knob 4 which bends a bending portion 2w (described later) of the insertion section 2 in a vertical direction; and a lateral bending operation knob 6 which bends the bending portion 2w in a lateral direction.

The operation section 3 includes: a fixing lever 5 which fixes a rotary position of the vertical bending operation knob 4; and a fixing knob 7 which fixes a rotary position of the lateral bending operation knob 6.

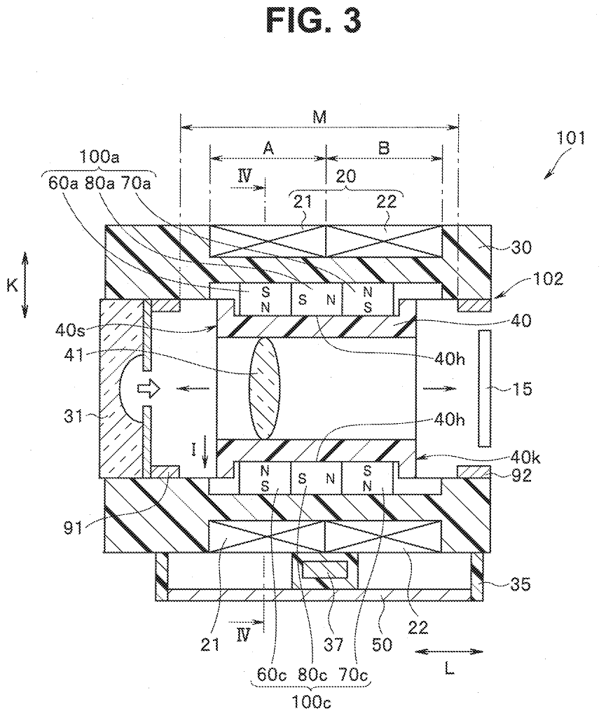

A zoom lever 10 which moves a movable barrel 40 of the drive unit 102 described later (both shown in FIG. 3) is disposed on the operation section 3.

The insertion section 2 includes a distal end portion 2s, the bending portion 2w, and a flexible tube portion 2k which are arranged in this order from a distal end side, and is formed in an elongated shape.

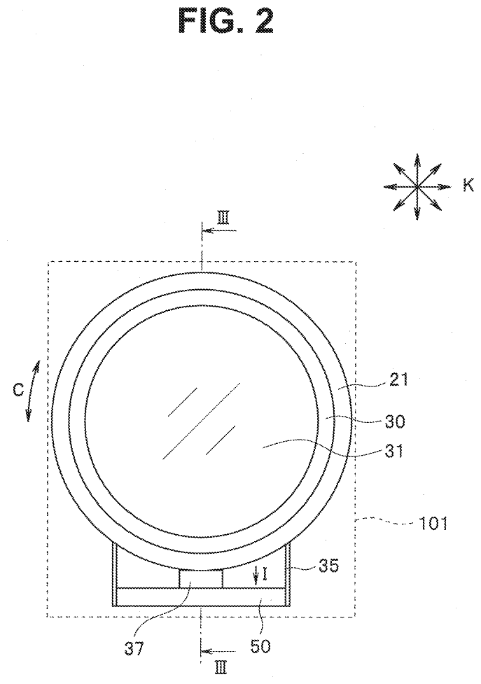

The bending portion 2w is bent in four directions, that is, in upward, downward, leftward, and rightward directions, for example, due to rotary operations of the vertical bending operation knob 4 and the lateral bending operation knob 6. With such operations, the bending portion 2w can change an observation direction of the image pickup apparatus 101 (see FIG. 2) described later which is disposed in the distal end portion 2s, and can enhance insertability of the distal end portion 2s in a subject. The flexible tube portion 2k is connected to a proximal end side of the bending portion 2w.

The image pickup apparatus 101 is disposed in the distal end portion 2s which is connected to a distal end side of the bending portion 2w.

Next, a configuration of the image pickup apparatus 101 and a configuration of the drive unit 102 are described with reference to FIG. 2 to FIG. 7.

FIG. 2 is a front view of the image pickup apparatus which is disposed in the distal end portion of the insertion section of the endoscope shown in FIG. 1. FIG. 3 is a cross-sectional view of the image pickup apparatus taken along a line III-III in FIG. 2.

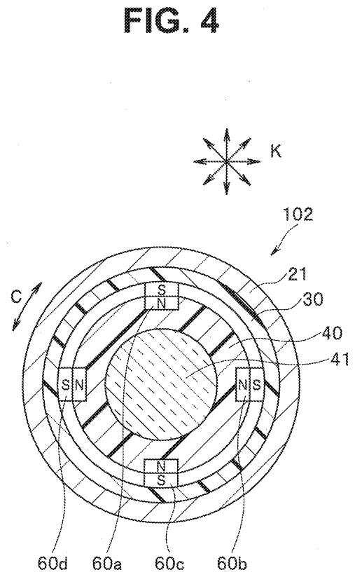

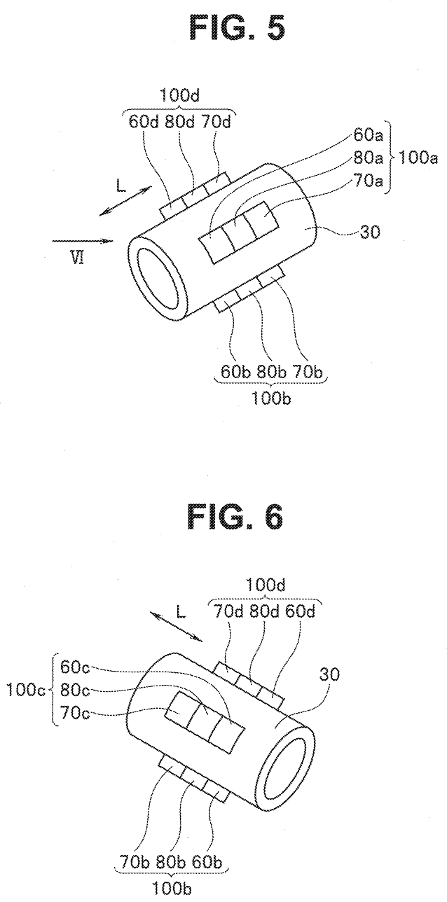

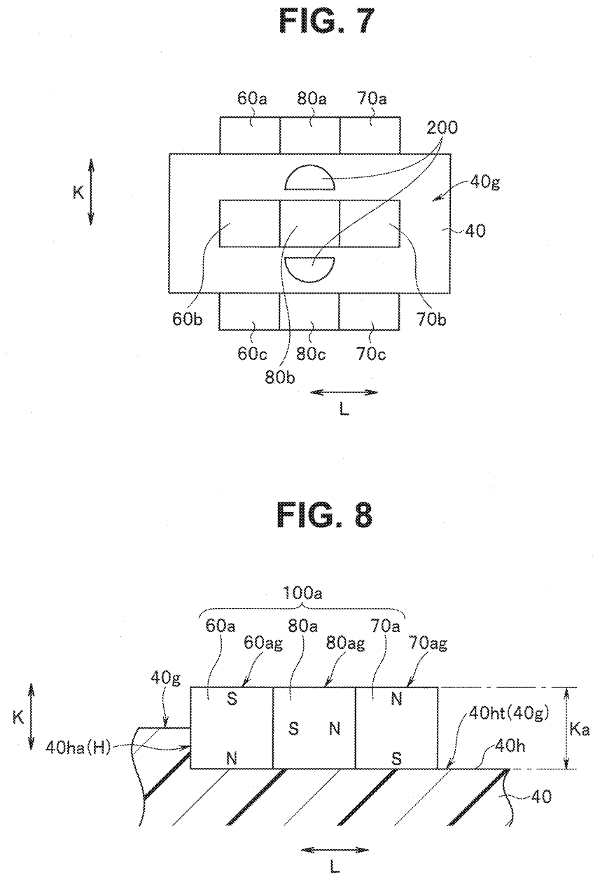

FIG. 4 is a cross-sectional view of the drive unit taken along a line IV-IV in FIG. 3. FIG. 5 is a perspective view schematically showing the movable barrel on which magnet units are disposed in the image pickup apparatus shown in FIG. 3. FIG. 6 is a perspective view of the movable barrel shown in FIG. 5 as viewed from a direction VI in FIG. 5. FIG. 7 is a side view showing a configuration where a rotation stopper is disposed on an outer peripheral surface of the movable barrel shown in FIG. 5.

As shown in FIG. 3, the image pickup apparatus 101 mainly includes an image pickup device 15 and a voice coil motor 102 which is a drive unit.

The voice coil motor 102 mainly includes a fixed barrel 30, the movable barrel 40, the magnet units 100a, 100b, 100c, and 100d, a coil unit 20, and movable range restricting members 91 and 92. It is not always necessary that the movable range restricting members 91 and 92 be independent members, and the movable range restricting members 91 and 92 may be integrally formed with the fixed barrel 30.

The movable barrel 40 holds a movable lens 41 which is an optical system for forming an optical image of an object in the movable barrel 40. The movable barrel 40 is movable in the fixed barrel 30 in an optical axis direction L of the movable lens 41.

As shown in FIG. 3 to FIG. 7, for example, four grooves 40h which extend in the optical axis direction L are formed on an outer peripheral surface 40g of the movable barrel 40 at an equal interval, for example, at every 90° in a circumferential direction C of the outer peripheral surface 40g.

The magnet units 100a, 100b, 100c, and 100d are disposed in the grooves 40h respectively. In other words, the magnet units 100a, 100b, 100c, and 100d are disposed, for example, at every 90° in the circumferential direction C.

As shown in FIG. 3 and FIG. 5, the magnet unit 100a is configured by three or more magnets which are disposed adjacently to each other in the optical axis direction L. For example, the magnet unit 100a is configured by three magnets including a first magnet 60a, a second magnet 70a, and a third magnet 80a.

As shown in FIG. 5 and FIG. 6, the magnet unit 100b is configured by three or more magnets which are disposed adjacently to each other in the optical axis direction L. For example, the magnet unit 100b is configured by three magnets including a first magnet 60b, a second magnet 70b, and a third magnet 80b.

As shown in FIG. 3 and FIG. 6, the magnet unit 100c is configured by three or more magnets which are disposed adjacently to each other in the optical axis direction L. For example, the magnet unit 100c is configured by three magnets including a first magnet 60c, a second magnet 70c, and a third magnet 80c.

As shown in FIG. 5 and FIG. 6, the magnet unit 100d is configured by three or more magnets which are disposed adjacently to each other in the optical axis direction L. For example, the magnet unit 100d is configured by three magnets including a first magnet 60d, a second magnet 70d, and a third magnet 80d.

The first magnets 60a to 60d are disposed so as not to be adjacent to the second magnets 70a to 70d in the optical axis direction L respectively, and the first magnets 60a to 60d and the second magnets 70a to 70d are polarized such that magnetic poles of the first magnets 60a to 60d and magnetic poles of the second magnets 70a to 70d are arranged differently from each other in a radial direction K of the movable barrel 40 orthogonal to the optical axis direction L.

More specifically, as shown in FIG. 3 and FIG. 4, in magnetization of the first magnets 60a to 60d, for example, an N pole is set at the inner side in the radial direction K and an S pole is set at the outer side in the radial direction K. In the magnetization of the second magnets 70a to 70d, for example, an S pole is set at the inner side in the radial direction K and an N pole is set at the outer side in the radial direction K.

Provided that the magnetic poles of the first magnets 60a to 60d and the magnetic poles of the second magnets 70a to 70d are arranged differently from each other, in magnetization of the first magnets 60a to 60d, for example, the S pole may be set at the inner side in the radial direction K and the N pole may be set at the outer side in the radial direction K, and in the magnetization of the second magnets 70a to 70d, for example, the N pole may be set at the inner side in the radial direction K and the S pole may be set at the outer side in the radial direction K.

In the first embodiment, the third magnets 80a to 80d are disposed adjacently to the first magnets 60a to 60d and the second magnets 70a to 70d in the optical axis direction L respectively.

The third magnets 80a to 80d are polarized in the optical axis direction L such that the magnetic poles of the third magnets 80a to 80d have the same polarities as the magnetic poles of the first magnets 60a to 60d at the outer side in the radial direction K and magnetic poles of the second magnets 70a to 70d at the outer side in the radial direction K.

More specifically, in the magnetization of the third magnets 80a to 80d, the S pole is set at first magnets 60a to 60d side which is a distal end side in the optical axis direction L, and the N pole is set at second magnets 70a to 70d side which is a proximal end side in the optical axis direction L.

Needless to say, the polarization state is inversed from a state described above depending on a magnetic pole state of the first magnet 60a to 60d at the outer side in the radial direction K and a magnetic pole state of the second magnet 70a to 70d at the outer side in the radial direction K.

As shown in FIG. 3, the fixed barrel 30 having a barrel shape holds an object lens 31 at a distal end in the optical axis direction L in the fixed barrel 30. The fixed barrel 30 holds the movable barrel 40 behind the object lens 31 in the optical axis direction L in a state where the movable barrel 40 is movable in an advancing and retracting manner in the optical axis direction L.

In FIG. 3, although the description is omitted for the sake of brevity, a gap is formed between an inner peripheral surface of the fixed barrel 30 and the outer peripheral surface 40g of the movable barrel 40 so as to allow the movement of the movable barrel 40 in the optical axis direction L.

The coil unit 20 is wound around an outer periphery of the fixed barrel 30. When a current is supplied to the coil unit 20, the energization acts on a magnetic field of the magnet units 100a to 100d so that the movable barrel 40 is moved in the optical axis direction L.

The coil unit 20 includes: a first coil 21 which faces the first magnets 60a to 60d in the radial direction K; and a second coil 22 which faces the second magnets 70a to 70d in the radial direction K. In an energized state of the coil unit 20, the current directions are inverted from each other between the first coil 21 and the second coil 22.

As shown in FIG. 3, the first coil 21 and the second coil 22 are wound around the outer periphery of the fixed barrel 30 in a state where the first coil 21 and the second coil 22 are disposed adjacently to each other in the optical axis direction L.

As shown in FIG. 3, within a movable range M of the movable barrel 40 in the fixed barrel 30, the first magnets 60a to 60d constantly face the first coil 21, and the second magnets 70a to 70d constantly face the second coil 22.

More specifically, the first coil 21 is wound around the outer periphery of the fixed barrel 30 with a length A in the optical axis direction L such that the first coil 21 overlaps with a movable range of the first magnets 60a to 60d in the optical axis direction L within the movable range M of the movable barrel 40.

The second coil 22 is wound around the outer periphery of the fixed barrel 30 with a length B in the optical axis direction L such that the second coil 22 overlaps with a movable range of the second magnets 70a to 70d in the optical axis direction L within the movable range M of the movable barrel 40.

On the outer periphery of the fixed barrel 30, the first coil 21 is wound on a distal end side relative to the second coil 22 in the optical axis direction L.

The first coil 21 and the second coil 22 are configured such that a direction of a current supplied to the first coil 21 and a direction of a current supplied to the second coil 22 are opposite to each other.

With such a configuration, when currents having different directions respectively are supplied to the first coil 21 and the second coil 22, since a magnetization direction of the first magnets 60a to 60d and a magnetization direction of the second magnets 70a to 70d are opposite to each other, a drive force generated in the first magnets 60a to 60d and a drive force generated in the second magnets 70a to 70d act in the same direction due to the Fleming's left-hand rule.

By switching the directions of currents which are supplied to the first coil 21 and the second coil 22, the movable barrel 40 moves in the fixed barrel 30 in an advancing direction or in a retracting direction in the optical axis direction L. Along with the movement of the movable barrel 40, a focal point of a photographic object in the endoscope 1 is switched.

The movable barrel 40 is movable in the advancing direction to a position where a distal end 40s which is an end portion of the movable barrel 40 comes into contact with the movable range restricting member 91 disposed on a proximal end side relative to the object lens 31 at a distal end side of an inner periphery of the fixed barrel 30.

The movable barrel 40 is movable in the retracting direction to a position where a rear end 40k which is an end portion of the movable barrel 40 comes into contact with the movable range restricting member 92 disposed on a distal end side relative to the image pickup device 15 at a proximal end side of the inner periphery of the fixed barrel 30.

In other words, the movable range restricting members 91 and 92 define the movable range M of the movable barrel 40 in the optical axis direction L, and regardless of the movement of the movable barrel 40 in the optical axis direction L, as described previously, the movable range restricting members 91 and 92 define a state where the first magnets 60a to 60d face the first coil 21 and a state where the second magnets 70a to 70d face the second coil 22.

As described previously, with respect to the first magnets 60a to 60d, the second magnets 70a to 70d, and the third magnets 80a to 80d, respective four magnets are arranged at an equal interval of 90° on the outer peripheral surface 40g in the circumferential direction C.

The reason is that a magnetic force which is applied to the first magnets 60a to 60d and the second magnets 70a to 70d from the first coil 21 and the second coil 22 having a circumferential shape is made uniform in the entire circumferential direction of the outer peripheral surface 40g, that is, in a plurality of directions which constitute the radial direction K.

Accordingly, by taking into account the above-mentioned effect, the magnet units may adopt a configuration where three magnet units are disposed on the outer peripheral surface 40g at an equal interval of approximately 120° in the circumferential direction C, five or more magnet units are disposed on the outer peripheral surface 40g at an equal interval in the circumferential direction C, or the magnet units are disposed circumferentially.

As described previously, the third magnets 80a to 80d are disposed adjacently to the first magnets 60a to 60d and the second magnets 70a to 70d in the optical axis direction L respectively.

The third magnets 80a to 80d are polarized in the optical axis direction L such that the magnetic poles of the third magnets 80a to 80d have the same polarities as the magnetic poles of the first magnets 60a to 60d at the outer side in the radial direction K and the magnetic poles of the second magnets 70a to 70d at the outer side in the radial direction K.

With such a configuration, the magnetic poles of the first magnets 60a to 60d at the outer side in the radial direction K and the magnetic poles of the second magnets 70a to 70d at the outer side in the radial direction K repel the magnetic poles of the third magnets 80a to 80d which face the above-mentioned magnetic poles at the outer side in the optical axis direction L.

Accordingly, when a current is supplied to the coil unit 20, a magnetic field which is generated at the outer side of the magnet units 100a to 100d in the radial direction K, that is, a magnetic field which is generated at a coil unit 20 side becomes stronger than a magnetic field which is generated at the inner side of the magnet units 100a to 100d in the radial direction K. In other words, magnetic flux density is increased and hence, a drive force for moving the movable barrel 40 in the optical axis direction L is increased.

More specifically, it has been understood that, with a provision of the third magnets 80a to 80d, a magnetic field which is generated at the outer side of the magnet units 100a to 100d in the radial direction K is, for example, twice as large as a magnetic field which is generated at the inner side of the magnet units 100a to 100d in the radial direction K. As a result, it has been understood that a drive force for moving the movable barrel 40 is also increased approximately twice as large as a drive force generated in the case where the third magnets 80a to 80d are not provided.

In other words, the third magnets 80a to 80d are provided for increasing a drive force for moving the movable barrel 40.

A configuration for moving the movable barrel 40 in the optical axis direction L using the first coil 21, the second coil 22, the first magnets 60a to 60d, the second magnets 70a to 70d, and the third magnets 80a to 80d is well known except for the description made above and hence, the detailed description of such a configuration is omitted.

As shown in FIG. 2 and FIG. 3, a magnetic member 50 which is an energizing plate is disposed outside the coil unit 20 in the radial direction K such that the magnetic member 50 faces any one of the magnet units 100a to 100d.

The magnetic member 50 faces the magnet unit 100c as shown in FIG. 3, for example. The magnetic member 50 is held by a holding member 35 fixed to the fixed barrel 30.

The magnetic member 50 applies an attracting force I to the magnet unit 100c in the radial direction K.

A portion of the outer peripheral surface 40g of the movable barrel 40 is brought into pressure contact with the inner peripheral surface of the fixed barrel 30 on a magnetic member 50 side due to the magnetic member 50. Accordingly, the movable barrel 40 moves in an advancing or retracting manner in the optical axis direction L in a state where the portion of the outer peripheral surface 40g is pressed to the inner peripheral surface of the fixed barrel 30 on the magnetic member 50 side.

Accordingly, backlash brought about by the movement of the movable barrel 40 in the optical axis direction L is prevented.

As shown in FIG. 2 and FIG. 3, a Hall device 37 which is a position detection member is disposed outside the coil unit 20 in the radial direction K such that the Hall device 37 faces any one of the magnet units 100a to 100d.

As shown in FIG. 3, the Hall device 37 is held by the fixed barrel 30 such that the Hall device 37 faces the magnet unit 100c, for example. The Hall device 37 detects a position of the movable barrel 40 in the optical axis direction L by detecting a magnetic field of the magnet unit 100c.

As shown in FIG. 7, a rotation stopper member 200 is disposed on the outer peripheral surface 40g of the movable barrel 40. The rotation stopper member 200 having a larger height than the magnet unit 100b in the radial direction K is disposed in a split manner in the circumferential direction C so as to sandwich the third magnet 80b of the magnet unit 100b in the circumferential direction C, for example.

The rotation stopper member 200 is fitted into grooves formed on an inner peripheral surface of the fixed barrel 30 in the optical axis direction L not shown. The rotation stopper member 200 is provided for preventing the movable barrel 40 from being rotated in the circumferential direction C along with the movement of the movable barrel 40 in the optical axis direction L.

The position at which the rotation stopper member 200 is disposed is not limited to the position shown in FIG. 7. The rotation stopper member 200 may be disposed on the outer peripheral surface 40g in a split manner in the circumferential direction C at the position where the rotation stopper member 200 sandwiches any one of the third magnets 80a to 80d.

By providing the rotation stopper member in this manner, the rotation of the movable barrel in the circumferential direction C is prevented so that stability of driving of the movable barrel is ensured. By forming the rotation stopper member in a split manner in the circumferential direction C and by sandwiching the magnet unit between split members of the rotation stopper member, it is possible to acquire both ensuring stability of driving of the movable barrel and enhancement of a drive force for driving the movable barrel with a small space.

Other components of the voice coil motor 102 and other components of the image pickup apparatus 101 are well known and hence, the description of such components is omitted.

In this manner, in the first embodiment, the description has been made with respect to the case where the magnet units 100a to 100d disposed on the outer peripheral surface 40g of the movable barrel 40 each are configured by at least three magnets, that is, the first magnets 60a to 60d, the second magnets 70a to 70d, and the third magnets 80a to 80d.

The description has been made with respect to the case where the first magnets 60a to 60d and the second magnets 70a to 70d are disposed so as not to adjacent to each other in the optical axis direction L respectively, and are polarized such that the magnetic poles of the first magnets 60a to 60d and the magnetic poles of the second magnets 70a to 70d are arranged differently from each other in the radial direction K.

The description has been made with respect to the case where the third magnets 80a to 80d are disposed adjacently to the first magnets 60a to 60d and the second magnets 70a to 70d in the optical axis direction L respectively, and these magnets is polarized in the optical axis direction L such that the magnetic poles of the third magnets 80a to 80d have the same polarities as the magnetic poles of the first magnets 60a to 60d at the outer side in the radial direction K and magnetic poles of the second magnets 70a to 70d at the outer side in the radial direction K.

The description has been made with respect to the case where the movable barrel 40 is movable in the fixed barrel 30 by the movable range M within the range that the coil unit 20 is disposed in the optical axis direction L.

With such configurations, when a current is supplied to the coil unit 20, a magnetic field which is generated at the outer side of the magnet units 100a to 100d in the radial direction K becomes stronger than a magnetic field generated at the inner side of the magnet units 100a to 100d in the radial direction K, that is, the magnetic flux density is increased.

Accordingly, a drive force for moving the movable barrel 40 within the movable range M in the optical axis direction L is increased compared to a case where the third magnets 80a to 80d are not provided.

Accordingly, a drive force for moving the movable barrel 40 can be increased without making the first magnets 60a to 60d and the second magnets 70a to 70d large in size in the radial direction K or without increasing the number of turns of the coil unit 20.

In other words, a drive force for moving the movable barrel 40 can be increased without making the voice coil motor 102 large-sized.

Accordingly, it is possible to provide the voice coil motor 102, the image pickup apparatus 101, and the endoscope 1 having a configuration by which a drive force for moving the movable barrel 40 can be ensured at the same level or more compared to the prior art, and the miniaturization can be realized.

### Second Embodiment

FIG. 8 is a partial cross-sectional view showing one example of a magnet unit disposed on an outer peripheral surface of a movable barrel in a drive unit according to a second embodiment.

A configuration of the drive unit according to the second embodiment differs from the drive unit according to the first embodiment described above and shown in FIG. 1 to FIG. 7 with respect to a point that magnet units are positioned with respect to the outer peripheral surface of the movable barrel by a position defining surface.

Accordingly, only such a different point is described, and components similar to the corresponding components in the first embodiment are given the same symbols, and the description of such components is omitted.

In the second embodiment described hereinafter, the magnet unit is described by taking a magnet unit 100a as an example. In other words, the configuration applied to the magnet unit 100a is also applicable to magnet units 100b to 100d.

As shown in FIG. 8, the magnet unit 100a is disposed in a state where the magnet unit 100a comes into contact with a bottom surface 40ht of a groove 40h formed on the outer peripheral surface 40g of the movable barrel 40.

The magnet unit 100a is fixed to the outer peripheral surface 40g such that surfaces 60ag, 70ag, and 80ag of a first magnet 60a, a second magnet 70a, and a third magnet 80a at an outer side in a radial direction K form a coplanar surface, that is, heights Ka of the first magnet 60a, the second magnet 70a, and the third magnet 80a in the radial direction become equal to each other.

A stepped portion 40ha which is a position defining surface H is formed on the outer peripheral surface 40g by the groove 40h. The magnet unit 100a butts against the stepped portion 40ha in the optical axis direction L.

The first magnet 60a comes into contact with the stepped portion 40ha in the optical axis direction L. Although not shown, the stepped portion formed by the groove 40h may be a stepped portion against which the second magnet 70a butts in the optical axis direction L.

With such a configuration, by making the magnet unit 100a butt against the position defining surface H in the optical axis direction L, the magnet unit 100a configured by the first magnet 60a, the third magnet 80a, and the second magnet 70a which are disposed adjacently to each other in the optical axis direction L is positioned on the outer peripheral surface 40g in the optical axis direction L.

Accordingly, irregularities in position of the magnet unit 100a in the optical axis direction can be reduced.

More specifically, in the same manner as described previously, within a movable range M of the movable barrel 40 in the optical axis direction L, the first magnet 60a and the second magnet 70a are positioned such that the first magnet 60a constantly faces a first coil 21, and the second magnet 70a constantly faces a second coil 22.

In this manner, irregularities in position of the magnet unit 100a in the optical axis direction can be reduced. Accordingly, irregularities in position of the first magnet 60a with respect to the first coil 21 in the optical axis direction, and irregularities in position of the second magnet 70a with respect to the second coil 22 in the optical axis direction can be reduced.

Other components of the second embodiment are equal to the corresponding components in the previously described first embodiment.

With such a configuration, within the movable range M of the movable barrel 40, a magnetic field of the first magnet 60a is applied to the first coil 21 which faces the first magnet 60a in the radial direction K without being leaked to the outside, and a magnetic field of the second magnet 70a is applied to the second coil 22 which faces the second magnet 70a in the radial direction K without being leaked to the outside and hence, the movable barrel 40 can be efficiently driven.

In other words, it is possible to prevent the occurrence of a phenomenon that the magnetic field of the first magnet 60a is applied to the second coil 22, and the magnetic field of the second magnet 70a is applied to the first coil 21 so that speed reduction force is applied to the movable barrel 40 from the coil unit 20 in a direction, in which the direction and an advancing direction in the optical axis direction L are opposite to each other.

Accordingly, a drive force loss caused by irregularities caused in assembling the magnet unit 100a to the outer peripheral surface 40g can be reduced and hence, a drive force which takes into account a drive force loss can also be designed to be small.

In view of the above, it is unnecessary to make a size of the magnet unit 100a larger than necessary or to increase the number of turns of the coil unit 20 and hence, it is possible to realize a miniaturization of the voice coil motor 102.

Other advantageous effects of the second embodiment are equal to the corresponding advantageous effects of the above-mentioned first embodiment.

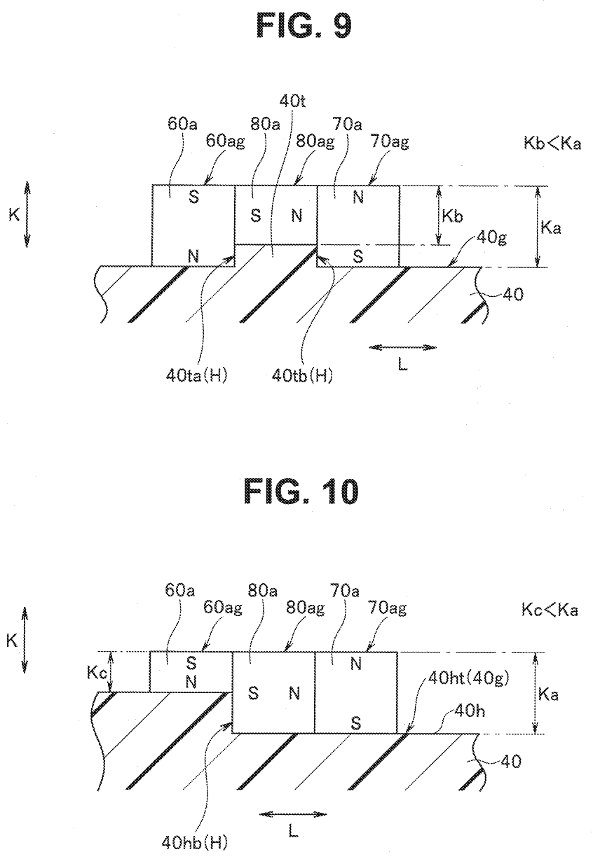

Hereinafter, modifications are described with reference to FIG. 9 and FIG. 10. FIG. 9 is a partial cross-sectional view showing a modification where the position defining surface shown in FIG. 8 is formed on a protruding portion formed on the outer peripheral surface of the movable barrel. FIG. 10 is a partial cross-sectional view showing a modification where the position defining surface shown in FIG. 8 is formed on a stepped portion against which the third magnet butts.

As shown in FIG. 9, the position defining surface H may be configured by end surfaces 40ta and 40tb of the protruding portion 40t formed on the outer peripheral surface 40g. The first magnet 60a and the second magnet 70a butt against the end surfaces 40ta and 40tb in the optical axis direction L respectively.

The third magnet 80a is placed on the protruding portion 40t. In a state where the third magnet 80a is placed on the protruding portion 40t, a height of the third magnet 80a in the radial direction K is set smaller than heights of the first magnet 60a and the second magnet 70a in the radial direction K (Kb<Ka) such that the heights Ka of the first magnet 60a, the second magnet 70a, and the third magnet 80a in the radial direction become equal to each other.

As shown in FIG. 10, the position defining surface H may be formed on the stepped portion 40hb which is formed by the groove 40h and against which the third magnet 80a butts in the optical axis direction L.

Also in this case, a height of the first magnet 60a in the radial direction K is set smaller than heights of the third magnet 80a and the second magnet 70a in the radial direction K (Kc<Ka) such that the heights Ka of the first magnet 60a, the second magnet 70a, and the third magnet 80a in the radial direction become equal to each other.

As has been described above, advantageous effects similar to the advantageous effects of the above-mentioned second embodiment can be acquired also by the position defining surfaces H shown in FIG. 9 and FIG. 10.

### Third Embodiment

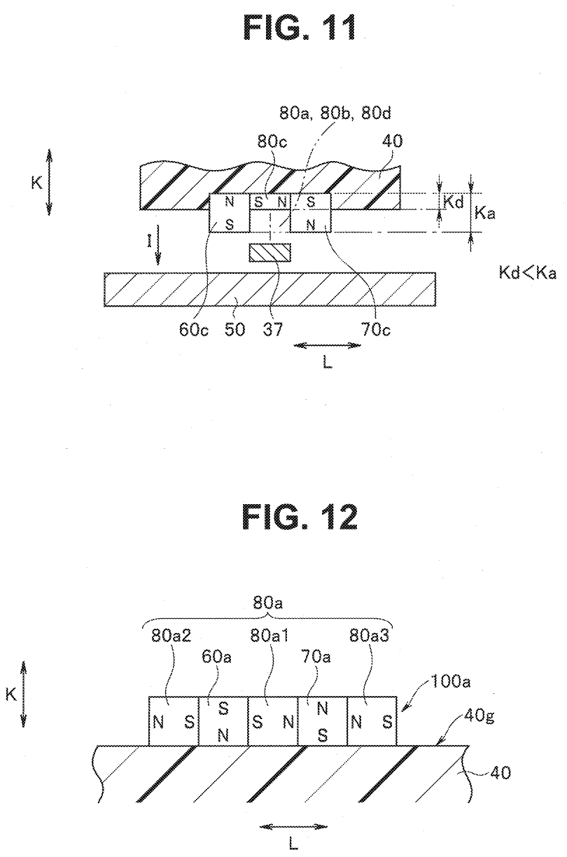

FIG. 11 is a partial cross-sectional view showing one example of a magnet unit disposed on an outer peripheral surface of a movable barrel in a drive unit according to a third embodiment together with a magnetic member and a Hall device.

A configuration of the drive unit according to the third embodiment differs from the configuration of the drive unit according to the first embodiment described above and shown in FIG. 1 to FIG. 7 with respect to a point that a height of a third magnet of a magnet unit which faces a magnetic member or a Hall device in a radial direction K is set smaller than heights of other third magnets in the radial direction K.

Accordingly, only such a different point is described, and components similar to the corresponding components in the first embodiment are given the same symbols, and the description of the components is omitted.

As shown in FIG. 11, among the magnet units 100a to 100d, a height Kd of the third magnet 80c in the radial direction K in the magnet unit 100c, for example, which faces the magnetic member 50 in the radial direction K is set smaller than heights Ka of third magnets 80a, 80b, and 80d, first magnets 60a to 60d, and second magnets 70a to 70d of other magnet units 100a, 100b, and 100d in the radial direction K (Kd<Ka).

An attracting force I generated by the magnetic member 50 becomes large due to a provision of the third magnet 80c compared to a case where the magnet unit 100c is configured by only the first magnet 60c and the second magnet 70c.

As a result, a slide resistance of the movable barrel 40 with respect to a fixed barrel 30 is increased thus giving rise to a drawback that a drive force for driving the movable barrel 40 is lowered.

However, with the configuration of the third embodiment described above, the attracting force I can be set to a proper value by setting a height of only the third magnet 80c which faces the magnetic member 50 small.

Accordingly, even when the configuration is adopted where backlash of the movable barrel 40 in the fixed barrel 30 is prevented using the magnetic member 50, the increase of a drive force for driving the voice coil motor 102 can be realized by miniaturizing the voice coil motor 102 due to the provision of the third magnets 80a to 80d.

The same goes for the Hall device 37. Only the third magnet 80c which faces the Hall device 37 in the radial direction K may be formed small in the same manner as the relationship between the magnetic member 50 and the third magnet 80c.

With such a configuration, also in a case where the increase of a drive force for driving the voice coil motor 102 is realized by miniaturizing the voice coil motor 102 due to the provision of the third magnets 80a to 80d, a magnetic field which the Hall device 37 detects can be set properly.

Accordingly, even when the third magnets 80a to 80d are provided to the voice coil motor 102, there is no possibility that position detection accuracy of the movable barrel 40 by the Hall device 37 is lowered.

Other components and advantageous effects of the third embodiment are equal to the corresponding components and advantageous effects of the first and second embodiments described above.

FIG. 12 is a partial cross-sectional view showing a modification where a coil unit is configured by five magnets.

In the first to third embodiments described above, the description has been made by taking the case where the magnet units 100a to 100d each include three magnets as an example.

In other words, in the first to third embodiments, the description has been made by taking the case where the magnet units 100a to 100d include one third magnet 80a, 80b, 80c, and 80d respectively, and such one third magnet 80a, 80b, 80c, and 80d is disposed adjacently to both the first magnet 60a, 60b, 60c, and 60d and the second magnet 70a, 70b, 70c, and 70d in the optical axis direction L, and is sandwiched between the first magnet 60a, 60b, 60c, and 60d and the second magnet 70a, 70b, 70c, and 70d, as an example.

The present invention is not limited to such embodiments. As shown in FIG. 12, each of the third magnets may be disposed adjacently to at least one of the first magnets 60a to 60d and the second magnets 70a to 70d in the optical axis direction L.

Accordingly, to take the magnet unit 100a as an example, the third magnet 80a may be configured by three magnets including a third magnet 80a1, a third magnet 80a2, and a third magnet 80a3.

The third magnet 80a1 is sandwiched between the first magnet 60a and the second magnet 70a in the optical axis direction L, and is disposed in contact with the first magnet 60a and the second magnet 70a.

The third magnet 80a2 is positioned on a distal end side relative to the first magnet 60a in the optical axis direction L, and is disposed in contact with the first magnet 60a.

The third magnet 80a3 is positioned on a proximal end side relative to the second magnet 70a in the optical axis direction L, and is disposed in contact with the second magnet 70a.

In other words, the magnet unit 100a may be configured by five magnets arranged in the optical axis direction L.

The description made above with respect to the magnet unit 100a is also similarly applicable to magnet units 100b to 100d.

In the above-mentioned first to third embodiments, the description has been made with respect to the case where the image pickup apparatus 101 having the voice coil motor 102 is provided to the endoscope 1. However, the present invention is not limited to such a configuration, and is also applicable to the case where the image pickup apparatus 101 is provided to miniaturized equipment besides an endoscope.

What is claimed is:

1. A drive unit comprising:
a fixed barrel having a barrel shape;
a movable barrel configured to hold an optical system which forms an optical image of an object, the movable barrel being movable in the fixed barrel in an optical axis direction of the optical system;
a plurality of magnet units disposed on an outer periphery of the movable barrel at an equal interval in a circumferential direction, each of the plurality of magnet units being configured by three or more magnets disposed adjacently to each other in the optical axis direction,
a first magnet and a second magnet in each of the magnet units, the first magnet and the second magnet being polarized such that magnetic poles of the first magnet and magnetic poles of the second magnet are arranged differently from each other in a radial direction of the movable barrel orthogonal to the optical axis direction, and being disposed so as not to be adjacent to each other in the optical axis direction;
a third magnet disposed adjacently to at least one of the first magnet and the second magnet in the optical axis direction and being polarized in the optical axis direction such that magnetic poles of the third magnet have same polarities as a magnetic pole of the first magnet at an outer side in the radial direction and a magnetic pole of the second magnet at the outer side in the radial direction;
a coil unit wound around an outer periphery of the fixed barrel, the coil unit being configured to move the movable barrel in the optical axis direction by generating a magnetic field applied to the magnet units;
a first coil and a second coil in the coil unit, a current direction of the first coil and a current direction of the second coil being inversed from each other in an energized state, the first coil facing the first magnet in the radial direction, and the second coil facing the second magnet in the radial direction; and
a movable range restricting member configured to allow both end portions of the movable barrel to come into contact with the movable range restricting member in the optical axis direction, the movable range restricting member being configured to define a state where the first magnet faces the first coil and a state where the second magnet faces the second coil regardless of movement of the movable barrel in the optical axis direction.

2. The drive unit according to claim 1, wherein the first coil and the second coil are disposed adjacently to each other in the optical axis direction, and
within a movable range of the movable barrel in the optical axis direction, the first magnet constantly faces the first coil, and the second magnet constantly faces the second coil.

3. The drive unit according to claim 1, wherein the plurality of magnet units come into contact with an outer peripheral surface of the outer periphery of the movable barrel, and
a position defining surface against which the magnet units butt in the optical axis direction is formed on the outer peripheral surface.

4. The drive unit according to claim 3, wherein the position defining surface is formed on a protruding portion which is formed on the outer peripheral surface of the movable barrel and against which the first magnet and the second magnet butt in the optical axis direction, and
in a state where the third magnet is placed on the protruding portion, a height of the third magnet in the radial direction is set smaller than heights of the first magnet and the second magnet in the radial direction such that the first magnet, the second magnet, and the third magnet have a same height in the radial direction.

5. The drive unit according to claim 3, wherein the first magnet, the second magnet, and the third magnet are disposed adjacently to each other in the optical axis direction, and the position defining surface is formed on a stepped portion which is formed on the outer peripheral surface of the movable barrel and against which the third magnet butts in the optical axis direction, and
in a state where the third magnet butts against the stepped portion, each of the plurality of magnet units is disposed on the outer peripheral surface such that heights of the first magnet, the second magnet, and the third magnet in the radial direction become equal to each other.

6. The drive unit according to claim 3, wherein the first magnet, the second magnet, and the third magnet are disposed adjacently to each other in the optical axis direction, and the position defining surface is formed on a stepped portion which is formed on the outer peripheral surface of the movable barrel and against which the first magnet or the second magnet butts in the optical axis direction, and
in a state where the first magnet or the second magnet butts against the stepped portion, each of the plurality of magnet units is disposed on the outer peripheral surface such that heights of the first magnet, the second magnet, and the third magnet in the radial direction become equal to each other.

7. The drive unit according to claim 1, wherein a rotation stopper member which restricts rotation of the movable barrel in the circumferential direction by coming into contact with the fixed barrel is disposed on an outer peripheral surface of the outer periphery of the movable barrel, and
the rotation stopper member is disposed so as to sandwich the third magnet in the circumferential direction.

8. The drive unit according to claim 1, further comprising an energizing plate disposed outside the coil unit in the radial direction, the energizing plate facing any one of the plurality of magnet units disposed on the outer periphery of the movable barrel in the radial direction, the energizing plate being configured to apply an attracting force to the magnet unit which the energizing plate faces,
wherein a height of the third magnet in the magnet unit which faces the energizing plate in the radial direction is set smaller than a height of the third magnet which does not face the energizing plate in the radial direction.

9. The drive unit according to claim 1, further comprising a position detection member disposed outside the plurality of magnet units in the radial direction, the position detection member facing any one of the plurality of magnet units disposed on the outer periphery of the movable barrel in the radial direction, the position detection member being configured to detect a position of the movable barrel by detecting a magnetic field of the magnet unit which the position detection member faces,
wherein a height of the third magnet in the magnet unit which faces the position detection member in the radial direction is set smaller than a height of the third magnet which does not face the position detection member in the radial direction.

10. An image pickup apparatus comprising the drive unit according to claim 1.

11. An endoscope comprising the image pickup apparatus according to claim 10.
